(19) United States
(12) Patent Application Publication
(10) Pub. No.: US 2024/0175013 A1
(43) Pub. Date: May 30, 2024

(54) BIALLELIC KNOCKOUT OF TRAC

(71) Applicant: EmendoBio Inc., Wilmington, DE (US)

(72) Inventors: Lior Izhar, Tel Aviv (IL); Rafi Emmanuel, Ramla (IL)

(73) Assignee: EmendoBio Inc., Wilmington, DE (US)

(21) Appl. No.: 18/257,176

(22) PCT Filed: Dec. 14, 2021
(86) PCT No.: PCT/US2021/063290

§ 371 Date: Jun. 13, 2023

Related U.S. Application Data
(60) Provisional application No. 63/147,168, filed on Feb. 8, 2021.
(60) Provisional application No. 63/125,200, filed on Dec. 14, 2020.

(51) Int. Cl. C12N 15/11; C12N 5/0783; C12N 9/22

(52) U.S. Cl. CPC C12N 15/111; C12N 5/0636; C12N 9/22; C12N 2310/20; C12N 2320/30

(57) ABSTRACT

RNA molecules comprising a guide sequence portion having 17-50 contiguous nucleotides containing nucleotides in the sequence set forth in any one of SEQ ID NOs: 1-1932 and compositions, methods, and uses thereof.

This application claims the benefit of U.S. Provisional Application Nos. 63/147,168, filed Feb. 8, 2021, and 63/125,200, filed Dec. 14, 2020, the contents of each of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “211214_91653-A-PCT_Sequence_Listing_AWG.txt”, which is 1,124 kilobytes in size, and which was created on Dec. 14, 2021 in the IBM-PC machine format, having an operating system compatibility with MS-Windows, which is contained in the text file filed Dec. 14, 2021 as part of this application.

### BACKGROUND OF INVENTION

Chimeric antigen receptors (CAR) provide a promising approach for immunotherapy. However, to make such therapies (e.g. CAR-T cell therapies) more accessible, it is highly desirable to develop an allogeneic adoptive transfer strategy in which universal CAR cells derived from cells from healthy donors can be applied to treat multiple patients. In order to implement this strategy, the αβ T-cell receptor (TCR) on allogenic CAR-T cells needs to be eliminated by knocking out T Cell Receptor Alpha Constant (TRAC) in order to avoid graft-versus-host-disease (GVHD). Additionally, to minimize the immunogenicity of CAR-T cells human leukocyte antigens class I (HLA-Is) may need to be removed by knocking out beta-2 microglobulin (B2M).

### SUMMARY OF THE INVENTION

Disclosed are approaches for knocking out the TRAC gene on CAR-T cells to avoid graft-versus-host-disease (GVHD) during allogeneic adoptive transfer. Accordingly, biallelic knockout of the TRAC gene in cells as described herein may be utilized to minimize their immunogenicity.

The present disclosure also provides a method for inactivating alleles of the T Cell Receptor Alpha Constant (TRAC) gene in a cell, the method comprising
introducing to the cell a composition comprising:
   a CRISPR nuclease or a sequence encoding the CRISPR nuclease; and
   an RNA molecule comprising a guide sequence portion having 17-50 nucleotides or a nucleotide sequence encoding the same,

wherein a complex of the CRISPR nuclease and the RNA molecule affects a double strand break in the allele of the TRAC gene.

According to embodiments of the present invention, there is provided an RNA molecule comprising a guide sequence portion having 17-50 contiguous nucleotides containing nucleotides in the sequence set forth in any one of SEQ ID Nos: 1-1932.

According to some embodiments of the present invention, there is provided a composition comprising an RNA molecule comprising a guide sequence portion having 17-50 contiguous nucleotides containing nucleotides in the sequence set forth in any one of SEQ ID NOs: 1-1932 and a CRISPR nuclease.

According to some embodiments of the present invention, there is provided a method for inactivating a TRAC allele in a cell, the method comprising delivering to the cell a composition comprising an RNA molecule comprising a guide sequence portion having 17-50 contiguous nucleotides containing nucleotides in the sequence set forth in any one of SEQ ID NOs: 1-1932 and a CRISPR nuclease. In some embodiments, the cell is a lymphocyte. In some embodiments, the cell is a T cell. In some embodiments, the cell is a T regulatory cell. In some embodiments, the cell is a stem cell. In some embodiments, the cell is an induced pluripotent stem cell (iPSC). In some embodiments, the cell is a fibroblast, blood cell, hepatocyte, keratinocyte, or any other cell type capable of being reprogrammed to an induced pluripotent stem cell (iPSC). In some embodiments, the delivering to the cell is performed in vivo, ex vivo, or in vitro. In some embodiments, the method is performed ex vivo and the cell is provided/explanted from an individual patient. In some embodiments, the method further comprises the step of introducing the resulting cell, with the modified/knocked out TRAC allele, into the individual patient or a different individual patient. Each possibility represents a separate embodiment. In some embodiments, the cell is originated from the individual patient to be treated. In some embodiments, the cell is originated from a donor. In some embodiments, the cell is allogeneic to the individual patient to which it is introduced.

According to some embodiments of the present invention, there is provided a method for treating and/or preventing graft-versus-host-disease (GVHD), the method comprising delivering to a cell of a subject having or at risk of experiencing graft-versus-host-disease (GVHD) a composition comprising an RNA molecule comprising a guide sequence portion having 17-50 contiguous nucleotides containing nucleotides in the sequence set forth in any one of SEQ ID NOs: 1-1932 and a CRISPR nuclease. In some embodiments, the method is for increasing persistence and/or engraftment of a cell in a host subject, the method comprising delivering to the cell a composition comprising an RNA molecule comprising a guide sequence portion having 17-50 contiguous nucleotides containing nucleotides in the sequence set forth in any one of SEQ ID NOs: 1-1932 and a CRISPR nuclease: and introducing the cell to the host subject. In some embodiments, the cell is further engineered to express a chimeric antigen receptor. In some embodiments, the cell is further engineered to knockout B2M gene to avoid host versus graft response following introduction into a host subject. In some embodiments, the cell is a stem cell or an iPSC or a progenitor cell and is differentiated to a T cell prior to introducing the cell to the host subject.

According to some embodiments of the present invention, there is provided use of a composition comprising an RNA molecule comprising a guide sequence portion having 17-50 contiguous nucleotides containing nucleotides in the sequence set forth in any one of SEQ ID NOs: 1-1932 and a CRISPR nuclease for inactivating a TRAC allele in a cell, comprising delivering to the cell the composition comprising an RNA molecule comprising a guide sequence portion having 17-50 contiguous nucleotides containing nucleotides in the sequence set forth in any one of SEQ ID NOs: 1-1932 and a CRISPR nuclease.

According to embodiments of the present invention, there is provided a medicament comprising an RNA molecule comprising a guide sequence portion having 17-50 contiguous nucleotides containing nucleotides in the sequence set forth in any one of SEQ ID NOs: 1-1932 and a CRISPR nuclease for use in inactivating a TRAC allele in a cell, wherein the medicament is administered by delivering to the cell the composition comprising an RNA molecule comprising a guide sequence portion having 17-50 contiguous nucleotides containing nucleotides in the sequence set forth in any one of SEQ ID NOs: 1-1932 and a CRISPR nuclease.

According to some embodiments of the present invention, there is provided use of a composition comprising an RNA molecule comprising a guide sequence portion having 17-50 contiguous nucleotides containing nucleotides in the sequence set forth in any one of SEQ ID NOs: 1-1932 and a CRISPR nuclease for treating, ameliorating, or preventing graft-versus-host-disease (GVHD), comprising delivering to a cell of a subject having or at risk of experiencing graft-versus-host-disease (GVHD) the composition of comprising an RNA molecule comprising a guide sequence portion having 17-50 contiguous nucleotides containing nucleotides in the sequence set forth in any one of SEQ ID NOs: 1-1932 and a CRISPR nuclease.

According to some embodiments of the present invention, there is provided a medicament comprising the composition comprising an RNA molecule comprising a guide sequence portion having 17-50 contiguous nucleotides containing nucleotides in the sequence set forth in any one of SEQ ID NOs: 1-1932 and a CRISPR nuclease for use in treating, ameliorating, or preventing graft-versus-host-disease (GVHD), wherein the medicament is administered by delivering to a cell of a subject having or at risk of experiencing graft-versus-host-disease (GVHD) the composition comprising an RNA molecule comprising a guide sequence portion having 17-50 contiguous nucleotides containing nucleotides in the sequence set forth in any one of SEQ ID NOs: 1-1932 and a CRISPR nuclease.

According to some embodiments of the present invention, there is provided a kit for inactivating a TRAC allele in a cell, comprising an RNA molecule comprising a guide sequence portion having 17-50 contiguous nucleotides containing nucleotides in the sequence set forth in any one of SEQ ID NOs: 1-1932, a CRISPR nuclease, and/or a tracrRNA molecule; and instructions for delivering the RNA molecule: CRISPR nuclease, and/or the tracrRNA to the cell

According to some embodiments of the present invention, there is provided a kit for treating or preventing graft-versus-host-disease (GVHD)in a subject, comprising an RNA molecule comprising a guide sequence portion having 17-50 contiguous nucleotides containing nucleotides in the sequence set forth in any one of SEQ ID NOs: 1-1932, a CRISPR nuclease, and/or a tracrRNA molecule; and instructions for delivering the RNA molecule; CRISPR nuclease, and/or the tracrRNA to a cell of a subject having or at risk of graft-versus-host-disease (GVHD).

### BRIEF DESCRIPTION OF THE DRAWINGS

FIG. 1: Percent editing of various OMNI nucleases with various RNA spacers, or guide sequence portions, which target the TRAC gene and are described in Table 5.

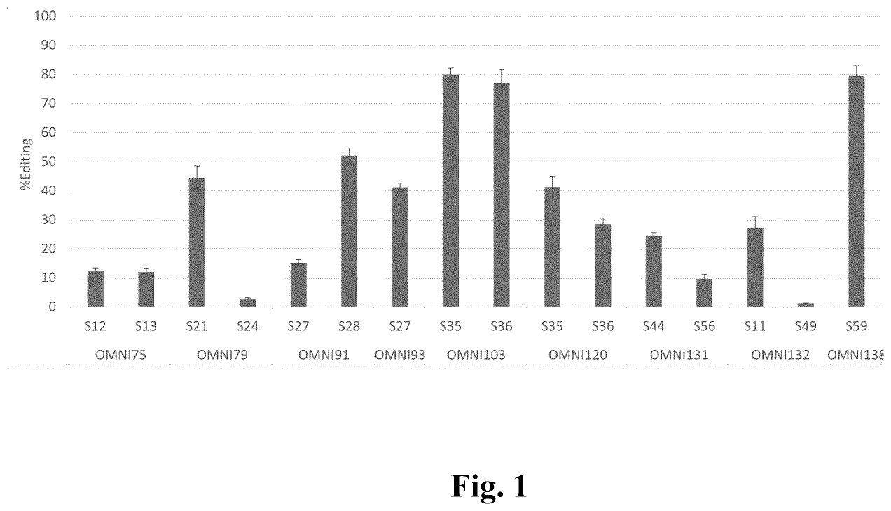

FIG. 2: Guide activity of OMNI-103 CRISPR nuclease guides targeting TRAC in HeLa cells. Specific guides were-co-transfected with OMNI-103 to determine their on-target activity. Cells were harvested 72 h post DNA transfection, genomic DNA was extracted, and the region of the mutation was amplified and analyzed by NGS. Transfection efficiency is measured by mCherry fluorescence. Bars represent % editing, dots represent % mCherry. The graph represents the % of editing±standard deviation (STDV) (performed in triplicate).

FIG. 3: OMNI-93 and OMNI-103 CRISPR nuclease TRAC editing activity as ribonucleoprotein (RNP) in U2OS a cell system. RNPs complexes of OMNI-93 and OMNI-103 and a specific guide molecule were electroporated into U2OS cells to determine their editing activity. Cells were harvested 72 h post RNP electroporation, genomic DNA was extracted, and the region of the mutation was amplified and analyzed by NGS. The graph represents the % of editing±STDV of two (2) independent electroporations.

FIG. 4: OMNI-103 CRISPR nuclease editing effect on protein levels of TRAC in primary T cells. RNPs complexes of OMNI-103 and a specific guide molecule were electroporated into activated primary T cells. Cells were harvested 72 hours days post RNP electroporation, and cells were collected and analyzed by a FACS assay.

### DETAILED DESCRIPTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

In some embodiments of the present invention, a DNA nuclease is utilized to affect a DNA break at a target site to induce cellular repair mechanisms, for example, but not limited to, non-homologous end-joining (NHEJ). During classical NHEJ, two ends of a double-strand break (DSB) site are ligated together in a fast but also inaccurate manner (i.e. frequently resulting in mutation of the DNA at the cleavage site in the form of small insertion or deletions).

As used herein, the term “modified cells” refers to cells in which a double strand break is affected by a complex of an RNA molecule and the CRISPR nuclease as a result of hybridization with the target sequence, i.e. on-target hybridization.

This invention provides a modified cell or cells obtained by use of any of the methods described herein. In an embodiment these modified cell or cells are capable of giving rise to progeny cells. In an embodiment these modified cell or cells are capable of giving rise to progeny cells after engraftment. As a non-limiting example, the modified cells may be hematopoietic stem cells (HSCs), or any cell suitable for an allogenic cell transplant or autologous cell transplant.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease, either alone or in combination with other RNA molecules, with the targeting sequence serving as the targeting portion of the CRISPR complex. When the molecule having the targeting sequence is present contemporaneously with the CRISPR nuclease, the RNA molecule, alone or in combination with an additional one or more RNA molecules (e.g. a tracrRNA molecule), is capable of targeting the CRISPR nuclease to the specific target sequence. As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferentially hybridizing a targeting sequence of a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments. the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-50, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. Preferably, the entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the RNA molecule having the guide sequence portion is present contemporaneously with the CRISPR molecule, alone or in combination with an additional one or more RNA molecules (e.g. a tracrRNA molecule), the RNA molecule is capable of targeting the CRISPR nuclease to the specific target DNA sequence. Accordingly, a CRISPR complex can be formed by direct binding of the RNA molecule having the guide sequence portion to a CRISPR nuclease or by binding of the RNA molecule having the guide sequence portion and an additional one or more RNA molecules to the CRISPR nuclease. Each possibility represents a separate embodiment. A guide sequence portion can be custom designed to target any desired sequence. Accordingly, a molecule comprising a “guide sequence portion” is a type of targeting molecule. In some embodiments, the guide sequence portion comprises a sequence that is the same as, or differs by no more than 1, 2, 3, 4, or 5 nucleotides from, a guide sequence portion described herein, e.g., a guide sequence set forth in any of SEQ ID NOs: 1-1932. Each possibility represents a separate embodiment. In some of these embodiments, the guide sequence portion comprises a sequence that is the same as a sequence set forth in any of SEQ ID NOs: 1-1932. Throughout this application, the terms “guide molecule,” “RNA guide molecule,” “guide RNA molecule,” and “gRNA molecule” are synonymous with a molecule comprising a guide sequence portion.

The term “non-discriminatory” as used herein refers to a guide sequence portion of an RNA molecule that targets a specific DNA sequence that is common to all alleles of a gene.

In embodiments of the present invention, an RNA molecule comprises a guide sequence portion having 17-50 contiguous nucleotides containing nucleotides in the sequence set forth in any one of SEQ ID NOs: 1-1932.

The RNA molecule and or the guide sequence portion of the RNA molecule may contain modified nucleotides. Exemplary modifications to nucleotides/polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of an RNA. An example of a modified polynucleotide is an mRNA containing 1-methyl pseudo-uridine. For examples of modified polynucleotides and their uses, see U.S. Pat. No. 8,278,036, PCT International Publication No. WO/2015/006747, and Weissman and Kariko (2015), each of which is hereby incorporated by reference.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be 17-50 nucleotides in length and contain 20-22 contiguous nucleotides in the sequence set forth in any one of SEQ ID NOs: 1-1932. In embodiments of the present invention, the guide sequence portion may be less than 22 nucleotides in length. For example, in embodiments of the present invention the guide sequence portion may be 17, 18, 19, 20, or 21 nucleotides in length. In such embodiments the guide sequence portion may consist of 17, 18, 19, 20, or 21 nucleotides, respectively, in the sequence of 17-22 contiguous nucleotides set forth in any one of SEQ ID NOs: 1-1932. For example, a guide sequence portion having 17 nucleotides in the sequence of 17 contiguous nucleotides set forth in SEQ ID NO: 1933 may consist of any one of the following nucleotide sequences (nucleotides excluded from the contiguous sequence are marked in strike-through):

(SEQ ID NO: 1933)
AAAAAAAUGUACUUGGUUCC
17 nucleotide guide sequence 1:
(SEQ ID NO: 1934) [symbol]
AAAUGUACUUGGUUCC
17 nucleotide guide sequence 2:
(SEQ ID NO: 1935) [symbol]
AAAAAUGUACUUGGUUC [symbol]
17 nucleotide guide sequence 3:
(SEQ ID NO: 1936) [symbol]
AAAAAAUGUACUUGGUU [symbol]
17 nucleotide guide sequence 4:
(SEQ ID NO: 1937)
AAAAAAAUGUACUUGGU

[symbol]

In embodiments of the present invention, the guide sequence portion may be greater than 20 nucleotides in length. For example, in embodiments of the present invention the guide sequence portion may be 21, 22, 23, 24, 25, 26, 27, 28, 29, or 30 nucleotides in length. In such embodiments the guide sequence portion comprises 17-50 nucleotides containing the sequence of 20, 21 or 22 contiguous nucleotides set forth in any one of SEQ ID NOs: 1-1932 and additional nucleotides fully complimentary to a nucleotide or sequence of nucleotides adjacent to the 3′ end of the target sequence, 5′ end of the target sequence, or both.

In embodiments of the present invention a CRISPR nuclease and an RNA molecule comprising a guide sequence portion form a CRISPR complex that binds to a target DNA sequence to effect cleavage of the target DNA sequence. CRISPR nucleases, e.g. Cpfl, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule, and a tracrRNA molecule. A guide sequence portion, which comprises a nucleotide sequence that is capable of hybridizing to a specific target DNA sequence, and a sequence portion that participates in CRIPSR nuclease binding, e.g. a tracrRNA sequence portion, can be located on the same RNA molecule. Alternatively, a guide sequence portion may be located on one RNA molecule and a sequence portion that participates in CRIPSR nuclease binding, e.g. a tracrRNA portion, may located on a separate RNA molecule. A single RNA molecule comprising a guide sequence portion (e.g. a DNA-targeting RNA sequence) and at least one CRISPR protein-binding RNA sequence portion (e.g. a tracrRNA sequence portion), can form a complex with a CRISPR nuclease and serve as the DNA-targeting molecule. In some embodiments, a first RNA molecule comprising a DNA-targeting RNA portion, which includes a guide sequence portion, and a second RNA molecule comprising a CRISPR protein-binding RNA sequence interact by base pairing to form an RNA complex that targets the CRISPR nuclease to a DNA target site or, alternatively, are fused together to form an RNA molecule that complexes with the CRISPR nuclease and targets the CRISPR nuclease to a DNA target site.

In embodiments of the present invention, an RNA molecule comprising a guide sequence portion may further comprise the sequence of a tracrRNA molecule. Such embodiments may be designed as a synthetic fusion of the guide portion of the RNA molecule and the trans-activating crRNA (tracrRNA). (See Jinek et al., 2012). In such an embodiment, the RNA molecule is a single guide RNA (sgRNA) molecule. Embodiments of the present invention may also form CRISPR complexes utilizing a separate tracrRNA molecule and a separate RNA molecule comprising a guide sequence portion. In such embodiments the tracrRNA molecule may hybridize with the RNA molecule via basepairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

According to embodiments of the present invention, there is provided a method for inactivating alleles of the T Cell Receptor Alpha Constant (TRAC) gene in a cell, the method comprising
introducing to the cell a composition comprising:
   at least one CRISPR nuclease, or a sequence encoding a CRISPR nuclease; and
   an RNA molecule comprising a guide sequence portion,

wherein a complex of the CRISPR nuclease and the RNA molecule affects a double strand break in alleles of the TRAC gene,

wherein the guide sequence portion of the RNA molecule comprises 17-50 contiguous nucleotides containing nucleotides in the sequence set forth in any one of SEQ ID NOs: 1-1932.

In some embodiments, the guide sequence portion of the RNA molecule comprises 17-50 contiguous nucleotides containing nucleotides in the sequence set forth in any one of SEQ ID NOs: 1-1538 modified to comprise up to five mismatches relative to the target site.

In some embodiments, the method is a method of preparing modified immune cells (e.g., T cells) for use in immunotherapy. In some embodiments, the method is performed in vitro or ex vivo.

In some embodiments, the composition is introduced to a cell in a subject or to a cell in culture.

In some embodiments, the cell is a lymphocyte, a T cell, a T regulatory cell, a stem cell, or a fibroblast, blood cell, hepatocyte, keratinocyte, or any other cell type capable of being reprogrammed to an induced pluripotent stem cell (iPSC).

In some embodiments, the cell is a hematopoietic stem cell (HSC), induced pluripotent stem cell (iPS cell), iPSc-derived cell, natural killer cell (NK), iPS-derived NK cell (iNK), T cell, innate-like T cell (iT), natural killer T cell (NKT), γ δ T cell, iPSc-derived T cell, invariant NKT cells (iNKT), iPSc-derived NKT, monocyte, or macrophage.

In some embodiments, the CRISPR nuclease and the RNA molecule are introduced to the cell at substantially the same time or at different times.

In some embodiments, alleles of the TRAC gene in the cell are subjected to an insertion or deletion mutation.

In some embodiments, the insertion or deletion mutation creates an early stop codon.

In some embodiments, the inactivating results in a truncated protein encoded by the mutated allele.

In some embodiments, the composition introduced to the cell further comprises a second RNA molecule comprising a guide sequence portion, wherein the guide sequence portion of the RNA molecule comprises 17-50 contiguous nucleotides containing nucleotides in the sequence set forth in any one of SEQ ID NOs: 1-1932.

According to embodiments of the present invention, there is provided a method for inactivating alleles of the T Cell Receptor Alpha Constant (TRAC) gene in a cell, the method comprising
introducing to the cell a composition comprising:
   at least one CRISPR nuclease, or a sequence encoding a CRISPR nuclease; and
   an RNA molecule comprising a guide sequence portion,

wherein a complex of the CRISPR nuclease and the RNA molecule affects a double strand break in alleles of the TRAC gene,

wherein the guide sequence portion of the RNA molecule comprises 17-50 contiguous nucleotides containing nucleotides in the sequence set forth in any one of SEQ ID NOs: 1-1932 modified to contain 1, 2, 3, 4, or 5 nucleotide mismatches relative to a fully-complementary target sequence of the guide sequence portion.

In some embodiments, the guide sequence portion of the RNA molecule comprises 1, 2, 3, 4, or 5 nucleotide mismatches relative to a fully-complementary target sequence of the guide sequence portion.

In some embodiments, the guide sequence portion having a mismatch provides higher targeting specificity to the complex of the CRISPR nuclease and the RNA molecule relative to a guide sequence portion that has higher complementarity to an allele of the TRAC gene.

In some embodiments, the composition introduced to the cell further comprises a second RNA molecule comprising a guide sequence portion, wherein the guide sequence portion of the RNA molecule comprises 17-50 contiguous nucleotides containing nucleotides in the sequence set forth in any one of SEQ ID NOs: 1-1932, or any one of SEQ ID NOs: 1-1932 modified to contain 1, 2, 3, 4, or 5 nucleotide mismatches relative to a fully-complementary target sequence of the guide sequence portion.

According to embodiments of the present invention, there is provided a composition comprising an RNA molecule which comprises 17-50 contiguous nucleotides containing nucleotides in the sequence set forth in any one of SEQ ID NOs: 1-1932.

In some embodiments, the RNA molecule comprises 17-50 contiguous nucleotides containing nucleotides in the sequence set forth in any one of SEQ ID NOs: 459, 482, 499, 608, 696, 719, 724, 1590, 1596, 1620, 1671, 1673, 1690 and 1723.

In some embodiments, the RNA molecule comprises 17-50 contiguous nucleotides containing nucleotides in the sequence set forth in any one of SEQ ID NOs: 1-1932 other than SEQ ID NOs: 459, 482, 499, 608, 696, 719, 724, 1590, 1596, 1620, 1671, 1673, 1690 and 1723.

In some embodiments, the composition further comprises a second RNA molecule which comprises 17-50 contiguous nucleotides containing nucleotides in the sequence set forth in any one of SEQ ID NOs: 1-1932, or any one of SEQ ID NOs: 1-1932 modified to contain 1, 2, 3, 4, or 5 nucleotide mismatches relative to a fully-complementary target sequence of the guide sequence portion.

In some embodiments, the second RNA molecule which comprises 17-50 contiguous nucleotides containing nucleotides in the sequence set forth in any one of SEQ ID NOs: 459, 482, 499, 608, 696, 719, 724, 1590, 1596, 1620, 1671, 1673, 1690 and 1723.

In some embodiments, the second RNA molecule which comprises 17-50 contiguous nucleotides containing nucleotides in the sequence set forth in any one of SEQ ID NOs: 1-1932 is other than SEQ ID NOs: 459, 482, 499, 608, 696, 719, 724, 1590, 1596, 1620, 1671, 1673, 1690 and 1723.

According to embodiments of the present invention, there is provided a composition comprising an RNA molecule which comprises 17-50 contiguous nucleotides containing nucleotides in the sequence set forth in any one of SEQ ID NOs: 1-1932, or any one of SEQ ID NOs: 1-1932 modified to contain 1, 2, 3, 4, or 5 nucleotide mismatches relative to a fully-complementary target sequence of the guide sequence portion.

In some embodiments, the composition further comprises a second RNA molecule which comprises 17-50 contiguous nucleotides containing nucleotides in the sequence set forth in any one of SEQ ID NOs: 1-1932, or any one of SEQ ID NOs: 1-1932 modified to contain 1, 2, 3, 4, or 5 nucleotide mismatches relative to a fully-complementary target sequence of the guide sequence portion.

In some embodiments, any one of the compositions described above further comprises a CRISPR nuclease.

In some embodiments, any one of the compositions described above further comprises a tracrRNA molecule.

According to embodiments of the present invention, there is provided a cell modified by any one of the methods described herein or modified using the any one of the compositions described herein. The modified cell may also have additional genes altered in order to improve their use for adoptive transfer, for example, reducing or preventing host-versus-graft disease (HvD).

In some embodiments, the cell is any one of a wherein the cell is a lymphocyte, a T cell, a T regulatory cell, a stem cell, or a fibroblast, blood cell, hepatocyte, keratinocyte, or any other cell type capable of being reprogrammed to an induced pluripotent stem cell (iPSC).

In some embodiments, the cell is a hematopoietic stem cell (HSC), induced pluripotent stem cell (iPS cell), iPSc-derived cell, natural killer cell (NK), iPS-derived NK cell (iNK), T cell, innate-like T cell (iT), natural killer T cell (NKT), γ δ T cell, iPSc-derived T cell, invariant NKT cell (iNKT), iPSc-derived NKT, monocyte, or macrophage.

In some embodiments, the cell is a stem cell or any cell type capable of being reprogrammed to an induced pluripotent stem cell (iPSC).

In some embodiments, the stem cell is differentiated after it is modified.

In some embodiments, the stem cell is differentiated into any one of a lymphocyte, a T cell, a T regulatory cell, a B cell, a natural killer (NK) cell, innate-like T cell (iT), natural killer T cells (NKT), γ δ T cell, invariant NKT cells (iNKT), monocyte, or macrophage.

According to embodiments of the present invention, there is provided a medicament comprising any one of the compositions described herein for use in inactivating a TRAC allele in a cell, wherein the medicament is administered by delivering to the cell the composition.

According to embodiments of the present invention, there is provided a use of any one of the compositions or modified cells described herein for treating, ameliorating, or preventing graft-versus-host-disease (GVHD), comprising delivering the composition or the modified cell to a subject experiencing or at risk of experiencing graft-versus-host-disease (GVHD).

According to embodiments of the present invention, there is provided a medicament comprising any one of the compositions or modified cells described herein for use in treating, ameliorating, or preventing graft-versus-host-disease (GVHD), wherein the medicament is administered by delivering the composition or the modified cell to a subject experiencing or at risk of experiencing graft-versus-host-disease (GVHD).

According to embodiments of the present invention, there is provided a kit for inactivating a TRAC allele in a cell, comprising any one of the compositions described herein and instructions for delivering the composition to the cell.

In some embodiments, wherein the composition is delivered to the cell ex vivo.

According to embodiments of the present invention, there is provided a kit for treating or preventing graft-versus-host-disease (GVHD) in a subject, comprising any one of the compositions or modified cells described herein and instructions for delivering the composition to a subject experiencing or at risk of experiencing graft-versus-host-disease (GVHD).

According to embodiments of the present invention, the modified immune cells (e.g., T cells) obtained by the described methods are intended to be used as a medicament for treating a cancer, infection, or immune disease in a subject in need thereof. The administration of the modified immune cell or a population thereof to a subject may be carried out in any convenient manner known in the art, including, but not limited to, aerosol inhalation, injection, ingestion, transfusion, implantation or transplantation. Injection or transfusion may be performed subcutaneously, intradermaliy, intratumorally, intranodally, intramedullary, intramuscularly, by intravenous or intralymphatic injection, or intraperitoneally. According to embodiments of the present invention, there is provided a method for adoptive cell therapy or prophylaxis comprising administering the modified cells to a subject suffering from or determined to be at risk of suffering from a cancer or an infection.

According to embodiments of the present invention, there is provided use of any one of the compositions or modified cells described herein for treating, ameliorating, or preventing graft-versus-host-disease (GVHD), comprising delivering the composition or modified cell to a subject experiencing or at risk of experiencing graft-versus-host-disease (GVHD).

According to embodiments of the present invention, there is provided a medicament comprising any one of the modified cells described herein for treating, ameliorating, or preventing graft-versus-host-disease (GVHD), such that the medicament is administered by delivering the composition to a subject experiencing or at risk of experiencing graft-versus-host-disease (GVHD).

According to embodiments of the present invention, there is provided an RNA molecule for use in modifying a cell (e.g. lymphocyte, T-cell, CAR-T cell) to treat, ameliorate, or prevent graft-versus-host-disease (GVHD) in a subject. The RNA molecule may be delivered to the cell ex vivo, in vitro, or in vivo.

According to embodiments of the present invention, there is provided a kit for inactivating a TRAC allele in a cell, comprising any one of the compositions described herein and instructions for delivering the composition to the cell.

According to embodiments of the present invention, there is provided a kit for treating or preventing graft-versus-host-disease (GVHD) in a subject, comprising any one of the compositions or modified cells described herein and instructions for delivering the composition or modified cell to a subject experiencing or at risk of experiencing graft-versus-host-disease (GVHD).

According to embodiments of the present invention, there is provided a cell modified by the method described herein or modified using the compositions described herein.

According to embodiments of the present invention, there is provided a gene editing composition comprising an RNA molecule comprising a guide sequence portion having 17-50 contiguous nucleotides containing nucleotides in the sequence set forth in any one of SEQ ID NOs: 1-1932. In some embodiments, the RNA molecule further comprises a portion having a sequence which binds to a CRISPR nuclease. In some embodiments, the sequence which binds to a CRISPR nuclease is a tracrRNA sequence.

In some embodiments, the RNA molecule further comprises a portion having a tracr mate sequence.

In some embodiments, the RNA molecule may further comprise one or more linker portions.

According to embodiments of the present invention, an RNA molecule may be up to 1000, 900, 800, 700, 600, 500, 450, 400, 350, 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 100 up to 300 nucleotides in length, 150 up to 300 nucleotides in length, 100 up to 500 nucleotides in length, 100 up to 400 nucleotides in length, 200 up to 300 nucleotides in length, 100 to 200 nucleotides in length, or 150 up to 250 nucleotides in length. Each possibility represents a separate embodiment.

According to some embodiments of the present invention, the composition further comprises a tracrRNA molecule.

According to some embodiments of the present invention, there is provided a method for inactivating TRAC expression in a cell, the method comprising delivering to the cell a composition comprising an RNA molecule comprising a guide sequence portion having 17-50 contiguous nucleotides containing nucleotides in the sequence set forth in any one of SEQ ID NOs: 1-1932 and a CRISPR nuclease.

According to some embodiments of the present invention, there is provided a method for preventing graft-versus-host-disease (GVHD), the method comprising delivering to a cell of a subject a composition comprising an RNA molecule comprising a guide sequence portion having 17-50 contiguous nucleotides containing nucleotides in the sequence set forth in any one of SEQ ID NOs: 1-1932 and a CRISPR nuclease.

According to embodiments of the present invention, at least one CRISPR nuclease and the RNA molecule or RNA molecules are delivered to the subject and/or cells substantially at the same time or at different times.

In some embodiments, a tracrRNA molecule is delivered to the subject and/or cells substantially at the same time or at different times as the CRISPR nuclease and RNA molecule or RNA molecules.

The compositions and methods of the present disclosure may be utilized for treating, preventing, ameliorating, or slowing progression of graft-versus-host-disease (GVHD).

Any one of, or combination of, the above-mentioned strategies for deactivating TRAC expression may be used in the context of the invention.

In embodiments of the present invention, an RNA molecule is used to direct a CRISPR nuclease to an exon or a splice site of a TRAC allele in order to create a double-stranded break (DSB), leading to insertion or deletion of nucleotides by inducing an error-prone non-homologous end-joining (NHEJ) mechanism and formation of a frameshift mutation in the TRAC allele. The frameshift mutation may result in, for example, inactivation or knockout of the TRAC allele by generation of an early stop codon in the TRAC allele and to generation of a truncated protein or to nonsense-mediated mRNA decay of the transcript of the allele. In further embodiments, one RNA molecule is used to direct a CRISPR nuclease to a promotor of a TRAC allele.

In some embodiments, the method is utilized for treating a subject at risk for graft-versus-host-disease (GVHD), which are disease phenotypes resulting from expression of the TRAC gene. In such embodiments, the method results in improvement, amelioration, or prevention of the disease phenotype.

Embodiments of compositions described herein include at least one CRISPR nuclease, RNA molecule(s), and a tracrRNA molecule, being effective in a subject or cells at the same time. The at least one CRISPR nuclease, RNA molecule(s), and tracrRNA may be delivered substantially at the same time or can be delivered at different times but have effect at the same time. For example, this includes delivering the CRISPR nuclease to the subject or cells before the RNA molecule and/or tracrRNA is substantially extant in the subject or cells.

In some embodiments, the cell is a lymphocyte. In some embodiments, the cell is a T cell. In some embodiments, the cell is a T regulatory cell. In some embodiments, the cell is a stem cell. In some embodiments, the cell is an induced pluripotent stem cell (iPSC). In some embodiments, the cell is a fibroblast, blood cell, hepatocyte, keratinocyte, or any other cell type capable of being reprogrammed to an induced pluripotent stem cell (iPSC).

### TRAC Editing Strategies

The present invention provides methods to knockout TRAC alleles in cells of a subject, thereby avoiding graft-versus-host-disease (GVHD) upon adoptive transfer of the cells or cells derived therefrom to a subject in need of the adoptive transfer. The provided methods to knockout TRAC alleles in a cell may be used to treat, prevent, or ameliorate graft-versus-host-disease (GVHD).

TRAC editing strategies include, but are not limited to, biallelic knockout by targeting any one of, or a combination of, Exons 1-3, including within seven nucleotides upstream and downstream of the exons to flank splice donor and acceptor sites, as frameshifts in these exons lead to non-functional, truncated TRAC proteins or non-sense mediated decay of the mutated TRAC transcripts.

### CRISPR Nucleases and PAM Recognition

In some embodiments, the sequence specific nuclease is selected from CRISPR nucleases, or is a functional variant thereof. In some embodiments, the sequence specific nuclease is an RNA guided DNA nuclease. In such embodiments, the RNA sequence which guides the RNA guided DNA nuclease (e.g., Cpfl) binds to and/or directs the RNA guided DNA nuclease to all TRAC alleles in a cell. In some embodiments, the CRISPR complex does not further comprise a tracrRNA. A skilled artisan will appreciate that RNA molecules can be engineered to bind to a target of choice in a genome by commonly known methods in the art.

The term “PAM” as used herein refers to a nucleotide sequence of a target DNA located in proximity to the targeted DNA sequence and recognized by the CRISPR nuclease complex. The PAM sequence may differ depending on the nuclease identity. In addition, there are CRISPR nucleases that can target almost all PAMs. In some embodiments of the present invention. a CRISPR system utilizes one or more RNA molecules having a guide sequence portion to direct a CRISPR nuclease to a target DNA site via Watson-Crick base-pairing between the guide sequence portion and the protospacer on the target DNA site, which is next to the protospacer adjacent motif (PAM), which is an additional requirement for target recognition. The CRISPR nuclease then mediates cleavage of the target DNA site to create a double-stranded break within the protospacer. In a non-limiting example, a type II CRISPR system utilizes a mature crRNA:tracrRNA complex that directs the CRISPR nuclease, e.g. Cas9 to the target DNA the target DNA via Watson-Crick base-pairing between the guide sequence portion of the crRNA and the protospacer on the target DNA next to the protospacer adjacent motif (PAM). A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example. NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant: NRRH for SpCas9-NRRH variant, wherein N is any nucleobase. R is A or G and H is A, C, or T; NRTH for SpCas9-NRTH variant, wherein N is any nucleobase, R is A or G and H is A, C, or T; NRCH for SpCas9-NRCH variant, wherein N is any nucleobase, R is A or G and H is A, C, or T; NG for SpG variant of SpCas9 wherein N is any nucleobase; NG or NA for SpCas9-NG variant of SpCas9 wherein N is any nucleobase; NR or NRN or NYN for SpRY variant of SpCas9, wherein N is any nucleobase, R is A or G and Y is C or T; NNG for Streptococcus canis Cas9 variant (ScCas9), wherein N is any nucleobase; NNNRRT for SaKKH-Cas9 variant of Staphylococcus aureus (SaCas9), wherein N is any nucleobase, and R is A or G: NNNNGATT for Neisseria meningitidis (NmCas9), wherein N is any nucleobase; TTN for Alicyclobacillus acidiphilus Cas12b (AacCas12b), wherein N is any nucleobase; or TTTV for Cpfl, wherein V is A, C or G. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break, either double or single-stranded in nature, at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Patent Publication No. 2015/0211023, incorporated herein by reference.

CRISPR systems that may be used in the practice of the invention vary greatly. CRISPR systems can be a type I, a type II, or a type III system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Casl0, Casl Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csxl7, Csxl4, Csxl0, Csxl6, CsaX, Csx3, Cszl, Csxl5, Csfl, Csf2, Csf3, Csf4, and Cul966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis,. Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii. Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculumthermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., SpCas9 D1135E variant, SpCas9 VQR variant, SpCas9 EQR variant, or SpCas9 VRER variant may also be used in the context of the invention.

Thus, an RNA guided DNA nuclease of a CRISPR system, such as a Cas9 protein or modified Cas9 or homolog or ortholog of Cas9, or other RNA guided DNA nucleases belonging to other types of CRISPR systems, such as Cpfl and its homologs and orthologs, may be used in the compositions of the present invention. Additional CRISPR nucleases may also be used, for example, the nucleases described in PCT International Application Publication Nos. WO2020/223514 and WO2020/223553, which are hereby incorporated by reference.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Derivatives include, but are not limited to, CRISPR nickases, catalytically inactive or “dead” CRISPR nucleases, and fusion of a CRISPR nuclease or derivative thereof to other enzymes such as base editors or retrotransposons. See for example, Anzalone et al. (2019) and PCT International Application No. PCT/US2020/037560.

Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpfl. Cpfl is a single RNA-guided endonuclease which utilizes a T-rich protospacer-adjacent motif. Cpfl cleaves DNA via a staggered DNA double-stranded break. Two Cpfl enzymes from Acidaminococcus and Lachnospiraceae have been shown to carry out efficient genome-editing activity in human cells. (See Zetsche et al., 2015).

Thus, an RNA guided DNA nuclease of a Type II CRISPR System, such as a Cas9 protein or modified Cas9 or homologs, orthologues, or variants of Cas9, or other RNA guided DNA nucleases belonging to other types of CRISPR systems, such as Cpfl and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, “beta, D-galactosylqueuosine”, 2′-O-methylguanosine, inosine, N6-isopentenyladenosine. 1-methyladenosine, I-methylpseudouridine. 1-methylguanosine, 1-methylinosine. “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-mannosylqueuosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-5-oxyacetic acid-methylester, uridine-5-oxyacetic acid, wybutoxosine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2′-O)-methyl-5-methyluridine, 2′-O-methyluridine, wybutosine, “3-(3-amino-3-carboxy-propyl)uridine, (acp3)u”, 2′-0-methyl (M), 3′-phosphorothioate (MS), 3′-thioPACE (MSP), pseudouridine, or 1-methyl pseudo-uridine. Each possibility represents a separate embodiment of the present invention.

In addition to targeting TRAC alleles by a RNA-guided CRISPR nuclease, other means of inhibiting TRAC expression in a target cell include but are not limited to use of a gapmer, shRNA, siRNA, a customized TALEN, meganuclease, or zinc finger nuclease, a small molecule inhibitor, and any other method known in the art for reducing or eliminating expression of a gene in a target cell. See, for example, U.S. Pat. Nos. 6,506,559; 7,560,438; 8,420,391; 8,552,171; 7,056,704; 7,078,196; 8,362,231; 8,372,968; 9,045,754; and PCT International Publication Nos. WO/2004/067736; WO/2006/097853; WO/2003/087341; WO/2000/0415661; WO/2003/080809; WO/2010/079430; WO/2010/079430; WO/2011/072246; WO/2018/057989; and WO/2017/164230, the entire contents of each of which are incorporated herein by reference.

Advantageously, the guide RNA molecules presented herein provide improved TRAC knockout efficiency when complexed with a CRISPR nuclease in a cell relative to other guide RNA molecules. These specifically designed sequences may also be useful for identifying TRAC target sites for other nucleotide targeting-based gene-editing or gene-silencing methods, for example, siRNA, TALENs, meganucleases or zinc-finger nucleases.

### Delivery to Cells

Any one of the compositions described herein may be delivered to a target cell by any suitable means. RNA molecule compositions of the present invention may be targeted to any cell which contains and/or expresses a TRAC allele, such as a mammalian lymphocyte or stem cell. For example, in one embodiment the RNA molecule specifically targets TRAC alleles in a target cell and the target cell is a lymphocyte, a T cell, a T regulatory cell, a B cell, a natural killer (NK) cell, a macrophage, a stem cell, or a fibroblast, blood cell, hepatocyte, keratinocyte, or any other cell type capable of being reprogrammed to an induced pluripotent stem cell (iPSC). The delivery to the cell may be performed in vivo, ex vivo, or in vitro. Further, the nucleic acid compositions described herein may be delivered to a cell as one or more of DNA molecules, RNA molecules, ribonucleoproteins (RNP), nucleic acid vectors, or any combination thereof.

In some embodiments, the RNA molecule comprises a chemical modification. Non-limiting examples of suitable chemical modifications include 2′-0-methyl (M), 2′-0-methyl, 3′phosphorothroate (MS) or 2′-0-methyl, 3 ‘thioPACE (MSP), pseudouridine, and 1-methyl pseudo-uridine. Each possibility represents a separate embodiment of the present invention.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992): Nabel & Felgner (1993): Mitani & Caskey (1993): Dillon (1993); Miller (1992): Van Brunt (1988): Vigne (1995): Kremer & Perricaudet (1995): Haddada et al. (1995): and Yu et al. (1994).

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus). (See, e.g., Chung et al., 2006). Sonoporation using, e.g., the Sonitron 2000 system (Rich-Mar), can also be used for delivery of nucleic acids. Cationic-lipid mediated delivery of proteins and/or nucleic acids is also contemplated as an in vivo. ex vivo, or in vitro delivery method. (See Zuris et al. (2015): see also Coelho et al. (2013): Judge et al. (2006); and Basha et al. (2011)).

Non-viral vectors, such as transposon-based systems e.g. recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa. RTM. Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam.TM., Lipofectin.TM. and Lipofectamine.TM. RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995): Blaese et al., (1995): Behr et al., (1994): Remy et al. (1994): Gao and Huang (1995): Ahmad and Allen (1992): U.S. Pat. Nos. 4,186,183: 4,217,344: 4,235,871: 4,261,975; 4,485,054: 4,501,728: 4,774,085: 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al., 2009).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992): Johann et al. (1992): Sommerfelt et al. (1990): Wilson et al. (1989): Miller et al. (1991): PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (See Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors. (Ellem et al., (1997): Dranoff et al., 1997).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, for example by systemic administration (e.g., intravitreal, intravenous, intraperitoneal, intramuscular, subdermal, or intracranial infusion) or topical application.

Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, optionally after selection for cells which have incorporated the vector. A non-limiting exemplary ex vivo approach may involve removal of tissue (e.g., peripheral blood, bone marrow; and spleen) from a patient for culture, nucleic acid transfer to the cultured cells (e.g., hematopoietic stem cells), followed by grafting the cells to a target tissue (e.g., bone marrow, and spleen) of the patient. In some embodiments, the stem cell or hematopoietic stem cell may be further treated with a viability enhancer.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney, “Culture of Animal Cells, A Manual of Basic Technique and Specialized Applications (6th edition, 2010) and the references cited therein for a discussion of how to isolate and culture cells from patients).

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

### Examples of RNA Guide Sequences which Specifically Target Alleles of TRAC Gene

Although a large number of guide sequences can be designed to target the TRAC gene, the nucleotide sequences described in Table 1 and are identified by SEQ ID NOs: 1-1932 were specifically selected to effectively implement the methods set forth herein.

Table 1 shows guide sequences designed for use as described in the embodiments above to associate with target sequences of the TRAC gene. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), SpRY (PAM SEQ: NRN or NYN), NmCas9WT (PAM SEQ: NNNNGATT), Cpfl (PAM SEQ: TTTV), or JeCas9WT (PAM SEQ: NNNVRYM). RNA molecules of the present invention are each designed to form complexes in conjunction with one or more different CRISPR nucleases and designed to target polynucleotide sequences of interest utilizing one or more different PAM sequences respective to the CRISPR nuclease utilized.

As used herein, the following nucleotide identifiers are used to represent a referenced nucleotide base(s):

| | |
|---|---|
| Nucleotide reference | Base(s) represented |

| | | | | |
|---|---|---|---|---|
| A | A | | | |
| C | | C | | |
| G | | | G | |
| T | | | | T |
| W | A | | | T |
| S | | C | G | |
| M | A | C | | |
| K | | | G | T |
| R | A | | G | |
| Y | | C | | T |
| B | | C | G | T |
| D | A | | G | T |
| H | A | C | | T |
| V | A | C | G | |
| N | A | C | G | T |

TABLE 1
Guide sequences designed to associate
with specific TRAC gene targets

| | SEQ ID | SEQ ID | SEQ ID |
|---|---|---|---|
| | NOs: of | NOs: of | NOs: of |
| | 20 base | 21 base | 22 base |
| Target | guides | guides | guides |
| 14:22547499-14:22547785 | 1-398 | 399-827 | 828-1286 |
| Exon 1, including the ATG start | | | |
| codon to 7nt DS of Exon 1 | | | |
| 14:22549631-14:22549689 | 1287-1333 | 1334-1384 | 1385-1437 |
| Exon 2, including 7nt US and | | | |
| 7nt DS to Exon 2 | | | |
| 14:22550550-14:22550671 | 1438-1586 | 1587-1747 | 1748-1911 |
| Exon 3, including 7nt US of | | | |
| Exon 3 to the stop codon | | | |

The indicated locations listed in column 1 of the Table 1 are based on gnomAD v3 database and UCSC Genome Browser assembly ID: hg38, Sequencing/Assembly provider ID: Genome Reference Consortium Human GRCh38.p12 (GCA_000001405.27). Assembly date: December 2013 initial release; December 2017 patch release 12.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Example 1: TRAC Modification Anaylsis

Guide sequences comprising 17-50 contiguous nucleotides containing nucleotides in the sequence set forth in any one of SEQ ID NOs: 1-1932 are screened for high on target activity using SpCas9 in T cells. On target activity is determined by DNA capillary electrophoresis analysis.

Additional examples demonstrating the editing ability of TRAC specific RNA guides are described below and may also be found in PCT International Publication No. WO 2020/223514 A2, the contents of which are hereby incorporated by reference.

### Example 2: OMNI-50 CRISPR Nuclease RNP Activity Using TRAC guides

TRAC editing by an OMNI-50 nuclease was observed in primary T cells, as can be seen in Table 2. 64% (22/34) of the tested TRAC guides were found to be active and had editing levels ranging between 5% to 84%.

19 TRAC guides displayed high editing. Considering the potential further optimization, full knock-out TRAC by OMNI-50 is possible with the appropriate strategy.

TABLE 2
OMNI-50 Activity as an RNP

| | Genomic | Corresponding | | % |
|---|---|---|---|---|
| System | site | spacer name | Spacer sequence | indels |
| Primary T | TRAC | gRNA 1 | UCUCUCAGCUGGUACACGGCA | 18% |
| cells | | | (SEQ ID NO: 1912) | |
| | | gRNA 2 | GCGUCAUGAGCAGAUUAAACC | 81% |
| | | | (SEQ ID NO: 1690) | |
| | | gRNA 3 | UCUCGACCAGCUUGACAUCAC | 10% |
| | | | (SEQ ID NO: 1913) | |
| | | gRNA 4 | UUAAACCCGGCCACUUUCAGG | 46% |
| | | | (SEQ ID NO: 1914) | |
| | | gRNA 5 | CUGUGCUAGACAUGAGGUCUA | 26% |
| | | | (SEQ ID NO: 608) | |
| | | gRNA 8 | ACUUCAAGAGCAACAGUGCUG | 3% |
| | | | (SEQ ID NO: 459) | |
| | | gRNA 9 | AAGAGCAACAGUGCUGUGGCC | 13% |
| | | | (SEQ ID NO: 1915) | |
| | | gRNA 10 | GCUGGGGAAGAAGGUGUCUUC | 7% |
| | | | (SEQ ID NO: 1916) | |
| | | gRNA 15 | AUAGGCAGACAGACUUGUCAC | 16% |
| | | | (SEQ ID NO: 499) | |
| | | gRNA 17 | UAGAGUCUCUCAGCUGGUACA | 23% |
| | | | (SEQ ID NO: 724) | |
| | | gRNA 18 | GUCUCUCAGCUGGUACACGGC | 5% |
| | | | (SEQ ID NO: 696) | |
| | | gRNA 19 | CAGCUGGUACACGGCAGGGUC | 11% |
| | | | (SEQ ID NO: 1917) | |
| | | gRNA 20 | AGCUGGUACACGGCAGGGUCA | 13% |
| | | | (SEQ ID NO: 482) | |
| | | gRNA 21 | UACACGGCAGGGUCAGGGUUC | 19% |
| | | | (SEQ ID NO: 719) | |
| | | gRNA 23 | CUUUCAAAACCUGUCAGUGAU | 4% |
| | | | (SEQ ID NO: 1673) | |
| | | gRNA 25 | UCCGAAUCCUCCUCCUGAAAG | 21% |
| | | | (SEQ ID NO: 1723) | |
| | | gRNA 26 | AAUCCUCCUCCUGAAAGUGGC | 11% |
| | | | (SEQ ID NO: 1596) | |
| | | gRNA 27 | AUCCUCCUCCUGAAAGUGGCC | 9% |
| | | | (SEQ ID NO: 1620) | |
| | | gRNA 29 | CUGCUCAUGACGCUGCGGCUG | 15% |
| | | | (SEQ ID NO: 1671) | |
| | | gRNA 30 | AGAUUAAACCCGGCCACUUUC | 24% |
| | | | (SEQ ID NO: 1918) | |
| | | gRNA 31 | AACCCGGCCACUUUCAGGAGG | 29% |
| | | | (SEQ ID NO: 1590) | |
| | | gRNA 32 | GCCACUUUCAGGAGGAGGAUU | 29% |
| | | | (SEQ ID NO: 1919) | |

OMNI-50 activity as RNP: OMNI-50 RNP was assembled with synthetic sgRNA (Agilent) and electroporated into cells. Several cell types were tested with a variety of sgRNAs. Cellular system, gene name, and spacer sequences are indicated next to the editing level as measured by NGS.

### Example 3: Multiplexing TRAC and B2M Editing

TRAC and B2M genes may be knocked out simultaneously by using a multiplexing approach. TRAC and B2M multiplex editing was tested by mixing two RNP populations and electroporating the mix into primary T cells. gRNA 32 was used for TRAC targeting and gRNA 15 was used for B2M targeting (spacer sequences are listed in Table 3). At 72 h cells were harvested and tested for editing by NGS. The TRAC gene measured 50% editing, and the B2M gene measured 25% editing. These results were similar to editing levels with a single RNP that was performed side-by-side to the multiplex test (Table 3).

TABLE 3
OMNI-50 Multiplexing

| | | | OMNI-50 Editing | | OMNI-50 STD | |
|---|---|---|---|---|---|---|
| Gene | Site | Spacer Sequence | Donor 1 | Donor 2 | Donor 1 | Donor 2 |
| TRAC | gRNA 32 | GCCACUUUCAG | 59.00 | 53.00 | 9.00 | 10.00 |
| | | GAGGAGGAUU | | | | |
| | | (SEQ ID NO: 1919) | | | | |
| B2M | gRNA 15 | UCACAGCCCAA | 42.00 | 44.00 | 13.00 | 19.00 |
| | | GAUAGUUAAG | | | | |
| | | (SEQ ID NO: 1938) | | | | |
| TRAC + | gRNA 32 + | Test for TRAC | 55.00 | 44.00 | 5.00 | 1.00 |
| B2M | gRNA 15 | | | | | |
| TRAC + | gRNA 32 + | Test for B2M | 22.00 | 27.00 | 3.00 | 1.00 |
| B2M | gRNA 15 | | | | | |

Table 3. OMNI-50 multiplexing in primary T cells: Multiplexing of OMNI-50 was performed by electroporation into activated primary T cells, targeting either TRAC or B2M genes, or combined targeting. The first two rows show each gene separately on two donors that were randomly chosen from a five-donor bank. The final two rows show the same analysis for each gene when electroporation was performed as a multiplex. Editing activity was determined by indel count after amplicon based NGS. Standard deviation of duplicates is also shown. Using only TRAC gRNA had no effect on the B2M gene and vice versa (not shown).

### Example 4: Evaluating Off-Targets Using a Guide-Seq Unbiased Analysis Method

To further evaluate the specificity of TRAC guides, the number of off-targets were tested across several sites using guide-seq as shown in Table 4 below.

TABLE 4
TRAC g32 off-targets

| | TRAC g32 Guide |
|---|---|
| SpCas9 #1 | 10 |
| SpCas9 #2 | 9 |
| OMNI-50 #1 | 5 |
| OMNI-50 #2 | 5 |
| SpCas9 on target editing | 93%, 81% |
| SpCas9 ODN integration | 14% |
| OMNI-50 on target editing | 51%, 74% |
| OMNI-50 ODN integration | 31%, 26% |

Table 4. TRAC off-target analysis by unbiased biochemical assay (guide seq): Off-target site counts of SpCas9 or OMNI-50 nucleases is shown in two replicates for the TRAC g32 guide. For this analysis, only amplified sites with ≥10 reads were analyzed, and sites with a lower number of reads were discarded in order to reduce background noise. The editing level at the on-target site determined by indel count after amplicon based NGS is also indicated, as well as ODN integration.

TABLE 5
Percent editing of various OMNI CRISPR Nucleases and TRAC-targeting spacers

| | Full Spacer | Spacer | Spacer | | | |
|---|---|---|---|---|---|---|
| Nuclease | Name | name | Sequence | PAM | Average | STD |
| OMNI-75 | OMN-175 site | S12 | SEQ ID | TCGGAA | 12.39 | 0.94 |
| | 12 | | NO: 1920 | | | |
| | OMNI-75 site | S13 | SEQ ID | GAGAGA | 12.12 | 1.13 |
| | 13 | | NO: 1921 | | | |
| OMNI-79 | OMNI-79 site | S21 | SEQ ID | CGGAAC | 44.4618344 | 3.99 |
| | 21 | | NO: 1922 | | | |
| | OMNI-79 site | S24 | SEQ ID | TGGACT | 2.80623815 | 0.29 |
| | 24 | | NO: 895 | | | |
| OMNI-91 | OMNI-91 site | S27 | SEQ ID | CAGGGTCA | 15.1033333 | 1.26666228 |
| | 27 | | NO: 1923 | | | |
| | OMNI-91 site | S28 | SEQ ID | CAGCGTCA | 51.9733333 | 2.71000615 |
| | 28 | | NO: 1793 | | | |
| OMNI-93 | OMNI-93 site | S27 | SEQ ID | CAGGGTCA | 41.1766667 | 1.42141948 |
| | 27 | | NO: 1923 | | | |
| OMNI-103 | OMNI-103 | S35 | SEQ ID | GAGACTCT | 79.8466667 | 2.33872472 |
| | S35 | | NO: 1080 | | | |
| | OMNI-103 | S36 | SEQ ID | CAGACTTG | 76.9666667 | 4.69017413 |
| | S36 | | NO: 1182 | | | |
| OMNI-120 | OMNI-120 35 | S35 | SEQ ID | GAGACTCT | 41.32 | 3.45131859 |
| | | | NO: 1080 | | | |
| | OMNI-120 | S36 | SEQ ID | CAGACTTG | 28.5166667 | 2.03524773 |
| | S36 | | NO: 1182 | | | |
| OMNI-131 | OMNI-131 | S44 | SEQ ID | GGAACCCA | 24.4933333 | 0.86932924 |
| | S44 | | NO: 1852 | | | |
| | OMNI-131 | S56 | SEQ ID | TGTACCAG | 9.63 | 1.50821086 |
| | S56 | | NO: 1924 | | | |
| OMNI-132 | OMNI-132 | S11 | SEQ ID | CTAAAT | 27.2766667 | 3.96389119 |
| | S11 | | NO: 1110 | | | |
| | OMNI-132 | S49 | SEQ ID | CAAAATCG | 1.27333333 | 0.09291573 |
| | S49 | | NO: 1925 | | | |
| OMNI-138 | OMNI-138 | S59 | SEQ ID | TCCAGTGA | 79.5966667 | 3.36295009 |
| | S59 | | NO: 1138 | | | |

TABLE 6
PAM sequences of OMNI nucleases present in TRAC Exons 1-4

| | PAM | | | | |
|---|---|---|---|---|---|
| Nuclease | Sequence | Exon 1 | Exon 2 | Exon 3 | Exon 4 |
| OMNI-91 | NNGNRY | 25 | 1 | 10 | 40 |
| | NNGNGTNA | 2 | 0 | 1 | 0 |
| OMNI-93 | NNGGD | 41 | 7 | 23 | 59 |
| | NNGGG | 0 | 0 | 2 | 5 |
| OMNI-103 | NNRRHY | 11 | 2 | 6 | 13 |
| | NNRACT | 4 | 1 | 1 | 3 |
| OMNI-120 | NRRAC | 11 | 2 | 6 | 13 |
| OMNI-138 | NNNNGBNH | 44 | 9 | 15 | 68 |
| OMNI-131 | NNNRCC | 12 | 1 | 8 | 25 |
| | NRNRCC | 6 | 0 | 5 | 10 |
| OMNI-132 | NNRAND | 38 | 7 | 12 | 59 |
| | NNAANT | 9 | 2 | 3 | 10 |

### Example 5: Additional OMNI CRISPR Nuclease Activity Assays Using TRAC Specific Guides

T cell receptors (TCR) recognize foreign antigens which have been processed as small peptides and bound to major histocompatibility complex (MHC) molecules at the surface of antigen presenting cells (APC). Each T cell receptor is a dimer consisting of one alpha and one beta chain or one delta and one gamma chain. TRAC encodes for the alpha subunit of the TCR.

In Hela cells, the CRIPSR nucleases listed in Table 8 were screened by introducing a nuclease and guide RNA molecule (Table 9) to a cell. Briefly, a screen was performed in 96-well format with 64 ng nuclease co-transfected with each of the guides at 20 ng using JetOPTIMUS reagent (Polyplus). Cells were harvested 72 hours post DNA transfection. Cell lysis and genomic DNA extraction was performed using Quick Extract (Lucigen) and endogenous genomic regions were amplified using specific primers to measure editing activity by next-generation sequencing (NGS). The screen identified 36 active novel nucleases. Editing was observed in 50 sites along the TRAC gene (Table 7). The sites for editing were chosen according to the PAM requirements for each nuclease, which was obtained by in vitro TXTL assay.

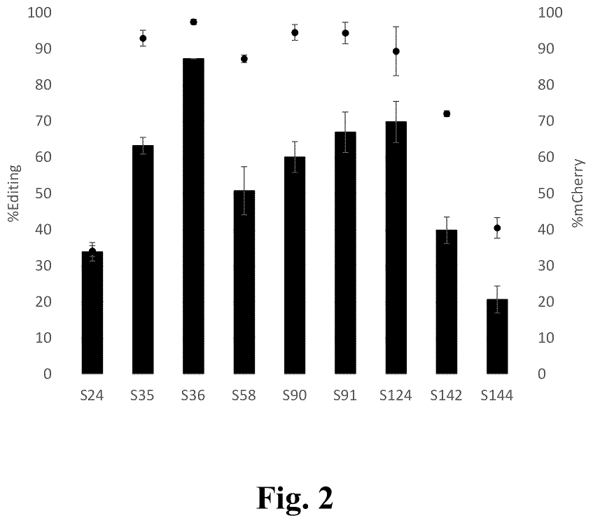

OMNI-103 CRISPR nuclease was further screened in Hela cells for additional sites along the TRAC gene. To this end, the corresponding OMNI-P2A-mCherry expression vector (Table 10) was transfected into HeLa cells together with a sgRNA molecule designed to target specific location in the TRAC gene (gRNA or “spacer” sequences are listed in Table 9). At 72 h, cells were harvested, and half of the cells were used for quantification of transfection efficiency by FACS using mCherry fluorescence as marker. The other half of the cells were lysed, and their genomic DNA content was used in PCR reaction that amplified the corresponding putative genomic targets. Amplicons were subjected to NGS and the resulting sequences were then used to calculate the percentage of editing events in each target site. Short insertions or deletions (indels) around the cut site are the typical outcome of repair of DNA ends following nuclease induced DNA cleavage. The calculation of % editing was therefore deduced from the fraction of indel-containing sequences within each amplicon. Significant editing activity was observed in nine (9) sites along the TRAC gene (FIG. 2).

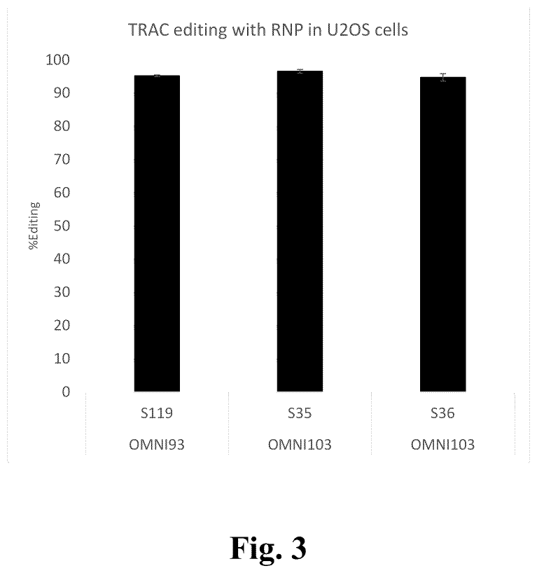

OMNI-93 and OMNI-103 were further developed for use as part of an RNP and tested in a U2OS cell line. Briefly, 2×10⁵ U2OS cells were mixed with preassembled RNPs composed of 105 pmole OMNI-93 or OMNI-103 protein and 120 pmole of a sgRNA comprising a 22-nucleotide guide sequence portion mixed with 100 pmole of electroporation enhancer (IDT-1075916). The mixture was electroporated into the cells using SE cell 4D-nucleofector X Kit S (V4XP-3032, Lonza) by applying the DN-100 program. OMNI-103 with no gRNA molecule served as a negative control. A fraction of the cells were harvested 72 hours post electroporation and genomic DNA was extracted to measure on-target activity by NGS. According to NGS analysis, all guides (FIG. 3) depicted indel activity ranging 90%-100%.

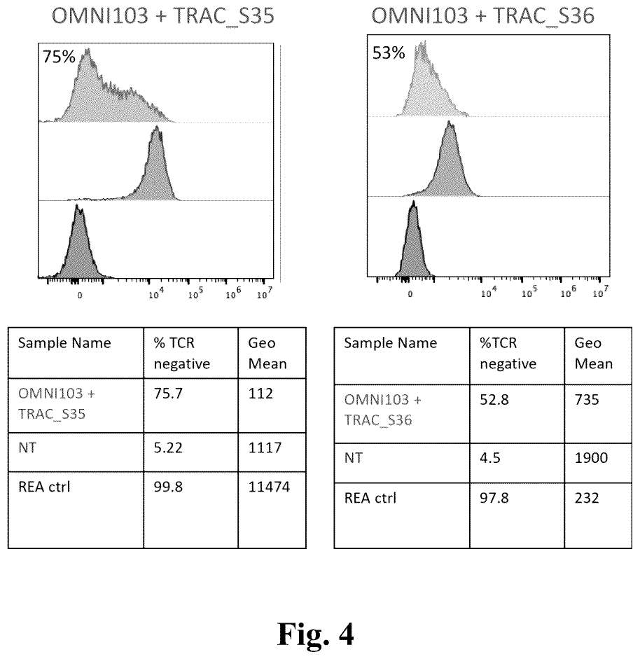

OMNI-103 was further tested as part of an RNP for TRAC editing in primary T cells using the same gRNA molecules showing editing activity in the U2OS cell system. Briefly, primary T cells were isolated from blood donations (Israel blood bank) using the buffy coat method for PBMC isolation, followed by negative isolation of CD3+ cells (T cell isolation kit, 130-096-535 Miltenyi). Primary T cells were then activated by CD3 and CD28 beads (T cell activation/expansion kit, 130-091-441 Miltenyi). 2×10⁵ primary T cells were mixed with preassembled RNPs composed of 113 pmole OMNI-103 protein and 160 pmole of a sgRNA having a 22-nucleotide guides sequence portion, and electroporated using P3 cell 4D-nucleofector X Kit S (V4XP-3032, Lonza) by applying the EH-115 program. A fraction of the cells were harvested 72 h post electroporation and TRAC surface expression was measured by flow cytometry (anti-TCR VioBlue, 1:50, #130-119-618 Miltenyi). According to FACS analysis, both guides S35 and S36 (FIG. 4) depicted editing activity ranging 50%-70%.

TABLE 7
OMNI CRISPR % Editing in HeLa Cells

| Gude Sequence | | | |
|---|---|---|---|
| Portion Name | Specific PAM | OMNI and PAM | % Editing |
| s12 | TCGGAA | OMNI-75 NNGNRA | 5.84 |
| s13 | GAGAGA | OMNI-75 NNGNRA | 10.54 |
| s24 | TGGACT | OMNI-79 NGG | 44.46 |
| s27 | CAGGGTCAGGGTTCTG | OMNI-91 NNGNGTNA | 15.10 |
| | GATA (SEQ ID NO: 2094) | | |
| s28 | GCGGTTATGGGCTTGC | OMNI-91 NNGNGTNA | 51.97 |
| | ATGT (SEQ ID NO: 2095) | | |
| s27 | CAGGGTCAGGGTTCTG | OMNI-93 NNGGG | 41.18 |
| | GATA (SEQ ID NO: 2096) | | |
| s36 | CAGACTTGTCACTGGA | OMNI-103 NNRACT | 79.85 |
| | TTTA (SEQ ID NO: 2097) | | |
| s35 | GAGACTCTAAATCCAG | OMNI-103 NNRACT | 76.97 |
| | TGAC (SEQ ID NO: 2098) | | |
| s21 | CGGAACCCAATCACTG | OMNI-104 NRRRH | 7.7 |
| | ACAG (SEQ ID NO: 2099) | | |
| s35 | GAGACTCTAAATCCAG | OMNI-120 NRVAC | 41.32 |
| | TGAC (SEQ ID NO: 2100) | | |
| s36 | CAGACTTGTCACTGGA | OMNI-120 NRVAC | 28.52 |
| | TTTA (SEQ ID NO: 2101) | | |
| s21 | CGGAAC | OMNI-124 NNGNRC | 25.62 |
| s35 | GAGACTCTAAATCCAG | OMNI-125 NARNC | 8.66 |
| | TGAC (SEQ ID NO: 2102) | | |
| s32 | AGGTAAGGGCAGCTTT | OMNI-126 NNNNAAG | 6.15 |
| | GGTG (SEQ ID NO: 2103) | | |
| s58 | CCAGCTGA | OMNI-127 NYAGC | 31.67 |
| s129 | ACAGCATT | OMNI-127 NYAGC | 11.97 |
| s130 | ACAGCCGC | OMNI-127 NYAGC | 60.58 |
| s44 | GGAACCCAATCACTGA | OMNI-131 NRNRCC | 24.49 |
| | CAGG (SEQ ID NO: 2104) | | |
| s11 | CTAAATCCAGTGACAA | OMNI-132 NNAANT | 27.28 |
| | GTCT (SEQ ID NO: 2105) | | |
| s59 | TCCAGTGACAAGTCTG | OMNI-138 NNNNGBNH | 79.60 |
| | TCTG (SEQ ID NO: 2106) | | |
| s34 | GTACCAGC | OMNI-159 NNNNCM | 16.74 |
| s34 | GTACCAGC | OMNI-169 NNRCCR | 16.34 |
| s101 | ACAAATCT | OMNI-173 NNAHRY | 46.25 |
| s14 | GGATAT | OMNI-173 NNAHRY | 30.50 |
| s20 | CGGCCA | OMNI-174 NNRC | 9.47 |
| s20 | CGGCCA | OMNI-175 NNRC | 8.32 |
| s20 | CGGCCA | OMNI-185 NNGM | 30.25 |
| s84 | GACGCTGC | OMNI-215 NVYGCT | 40.50 |
| s128 | AGGGTCAG | OMNI-218 NGGRKC | 65.40 |
| s22 | TGGAGC | OMNI-218 NGGRKC | 26.97 |
| s35 | GAGACTCT | OMNI-231 NVNRC | 34.19 |
| s62 | GAAACAGG | OMNI-231 NVNRC | 24.09 |
| s35 | GAGACTCT | OMNI-247 NRRRC | 8.75 |
| s35 | GAGACTCT | OMNI-250 NRNRC | 10.79 |
| s36 | CAGACTTG | OMNI-250 NRNRC | 10.40 |
| s160 | GACTCTAA | OMNI-266 NNNNCWMA | 50.61 |
| s72 | CAGCCCAG | OMNI-269 NNNNCM | 43.17 |
| s25 | CAGGGT | OMNI-274 NNRKGH | 25.67 |
| s52 | CCAGGTAA | OMNI-274 NNRKGH | 7.97 |
| s35 | GAGACTCT | OMNI-281 NNGR | 62.66 |
| s41 | CAGAAGAC | OMNI-281 NNGR | 86.30 |
| s56 | TGTACCAG | OMNI-293 NNNNCNA | 81.78 |
| s20 | CGGCCA | OMNI-295 NNRNCD | 8.23 |
| s135 | ATGTGTAT | OMNI-308 NNNNRYAT | 91.19 |
| s136 | CTTTGCAT | OMNI-308 NNNNRYAT | 34.72 |
| s95 | CCGTGTAC | OMNI-309 NNNNRWD | 7.08 |
| s74 | AATCCTCC | OMNI-316 NNNNCHY | 0.86 |
| s92 | AGAACCCT | OMNI-316 NNNNCHY | 32.85 |
| s95 | CCGTGTAC | OMNI-322 NNNNGNNC | 12.15 |
| s31 | TGAAACAG | OMNI-339 NRNAHC | 6.54 |
| s56 | TGTACCAG | OMNI-339 NRNAHC | 17.85 |
| s19 | GAACCC | OMNI-344 NNNCCC | 2.90 |
| s71 | TGACCCTG | OMNI-344 NNNCCC | 4.50 |
| s168 | AGCACAGT | OMNI-354 NRHACA | 8.45 |
| s35 | GAGACTCT | OMNI-358 NNRACY | 20.00 |
| s184 | GGTACACG | OMNI-366 NVTAM | 7.87 |
| s56 | TGTACCAG | OMNI-366 NVTAM | 4.20 |
| s35 | GAGACTCT | OMNI-368 NNNACT | 46.27 |
| s36 | CAGACTTG | OMNI-368 NNNACT | 14.88 |
| s40 | CAAAACTG | OMNI-369 NNNADC | 8.69 |
| s89 | AGCAACAG | OMNI-369 NNNADC | 16.42 |
| s69 | AGAAAAGC | OMNI-370 NNNAAA | 11.21 |
| s35 | GAGACTCT | OMNI-373 NRRAC | 3.10 |
| s92 | AGAACCCT | OMNI-375-NNAACNY | 46.10 |
| s93 | TAAACCCG | OMNI-375-NNAACNY | 4.10 |
| s180 | TTTAATCT | OMNI-376 NNYAA | 48.02 |
| s89 | AGCAACAG | OMNI-376 NNYAA | 1.35 |
| s56 | TGTACCAG | OMNI-377 NRDACC | 40.70 |
| s115 | GTGCTAGA | OMNI-378-NNG | 8.90 |
| s106 | ACCGATTT | OMNI-381 NNNGATD | 5.64 |
| s15 | GAGGAT | OMNI-381 NNNGATD | 16.72 |

TABLE 8
OMNI CRISPR Nuclease Elements

| | | SEQ ID NO | SEQ ID NO of | |
|---|---|---|---|---|
| OMNI | SEQ ID NO | of DNA | DNA sequence | OMNI |
| CRISPR | of OMNI | sequence | codon optimized | sgRNA |
| Nuclease | Amino Acid | encoding | for expression in | Scaffold |
| Name | Sequence | OMNI | human cells | Sequence |
| OMNI-75 | 1939 | 1975 | 2011 | 2047 |
| OMNI-79 | 1940 | 1976 | 2012 | 2048 |
| OMNI-91 | 1941 | 1977 | 2013 | 2049 |
| OMNI-93 | 1942 | 1978 | 2014 | 2050 |
| OMNI-103 | 1943 | 1979 | 2015 | 2051 |
| OMNI-104 | 1944 | 1980 | 2016 | 2052 |
| OMNI-120 | 1945 | 1981 | 2017 | 2053 |
| OMNI-124 | 1946 | 1982 | 2018 | 2054 |
| OMNI-125 | 1947 | 1983 | 2019 | 2055 |
| OMNI-126 | 1948 | 1984 | 2020 | 2056 |
| OMNI-127 | 1949 | 1985 | 2021 | 2057 |
| OMNI-131 | 1950 | 1986 | 2022 | 2058 |
| OMNI-132 | 1951 | 1987 | 2023 | 2059 |
| OMNI-138 | 1952 | 1988 | 2024 | 2060 |
| OMNI-159 | 1953 | 1989 | 2025 | 2061 |
| OMNI-169 | 1954 | 1990 | 2026 | 2062 |
| OMNI-173 | 1955 | 1991 | 2027 | 2063 |
| OMNI-174 | 1956 | 1992 | 2028 | 2064 |
| OMNI-175 | 1957 | 1993 | 2029 | 2065 |
| OMNI-185 | 1958 | 1994 | 2030 | 2066 |
| OMNI-215 | 1959 | 1995 | 2031 | 2067 |
| OMNI-218 | 1960 | 1996 | 2032 | 2068 |
| OMNI-231 | 1961 | 1997 | 2033 | 2069 |
| OMNI-247 | 1962 | 1998 | 2034 | 2070 |
| OMNI-250 | 1963 | 1999 | 2035 | 2071 |
| OMNI-266 | 1964 | 2000 | 2036 | 2072 |
| OMNI-269 | 1965 | 2001 | 2037 | 2073 |
| OMNI-274 | 1966 | 2002 | 2038 | 2074 |
| OMNI-281 | 1967 | 2003 | 2039 | 2075 |
| OMNI-293 | 1968 | 2004 | 2040 | 2076 |
| OMNI-295 | 1969 | 2005 | 2041 | 2077 |
| OMNI-308 | 1970 | 2006 | 2042 | 2078 |
| OMNI-309 | 1971 | 2007 | 2043 | 2079 |
| OMNI-322 | 1972 | 2008 | 2044 | 2080 |
| OMNI-339 | 1973 | 2009 | 2045 | 2081 |
| OMNI-354 | 1974 | 2010 | 2046 | 2082 |

TABLE 9
Guide Sequence Portion (“Spacer”) Elements

| | | PAM Site | Compatible OMNI |
|---|---|---|---|
| Spacer Name | Spacer Sequence | Utilized | CRISPR Nucleases |
| TRAC-S11 | GCCGUGUACCAGCUGAGAG | CTAAAT | OMNI-132 |
| | ACU (SEQ ID NO: 1110) | | |
| TRAC-S12 | CGGCCACUUUCAGGAGGAG | TCGGAA | OMNI-75 |
| | GAU (SEQ ID NO: 1920) | | |
| TRAC-S13 | CUGACCCUGCCGUGUACCA | GAGAGA | OMNI-75 |
| | GCU (SEQ ID NO: 1921) | | |
| TRAC-S15 | UAAACCCGGCCACUUUCAG | GAGGAT | |
| | GAG (SEQ ID NO: 1926) | | |
| TRAC-S19 | CCACUUUCAGGAGGAGGAU | GAACCC | |
| | UCG (SEQ ID NO: 1806) | | |
| TRAC-S20 | AGCGUCAUGAGCAGAUUAA | CGGCCA | OMNI-174, OMNI-175, |
| | ACC (SEQ ID NO: 1777) | | OMNI-185, OMNI-295 |
| TRAC-S21 | GGCCACUUUCAGGAGGAGG | CGGAAC | OMNI-104, OMNI-124 |
| | AUU (SEQ ID NO: 1922) | | |
| TRAC-S22 | CAAGAGCAACAGUGCUGUG | TGGAGC | OMNI-218 |
| | GCC (SEQ ID NO: 1927) | | |
| TRAC-S24 | ACUGUGCUAGACAUGAGGU | TGGACTTC | OMNI-103, OMNI-79 |
| | CUA (SEQ ID NO: 895) | | |
| TRAC-S25 | CUCAGCUGGUACACGGCAG | CAGGG | OMNI-274 |
| | GGU (SEQ ID NO: 1928) | | |
| TRAC-S27 | GAGUCUCUCAGCUGGUACA | CAGGGTCA | OMNI-91, OMNI-93 |
| | CGG (SEQ ID NO: 1923) | | |
| TRAC-S28 | CACCUCAGCUGGACCACAG | CAGCGTCA | OMNI-91 |
| | CCG (SEQ ID NO: 1793) | | |
| TRAC-S31 | UCAAGCUGGUCGAGAAAAG | TGAAACAG | OMNI-339 |
| | CUU (SEQ ID NO: 1421) | | |
| TRAC-S32 | AAGACACCUUCUUCCCCAG | AGGTAAGG | OMNI-126 |
| | CCC (SEQ ID NO: 847) | | |
| TRAC-S34 | UCCAGAACCCUGACCCUGC | GTACCAGC | OMNI-159, OMNI-169, |
| | CGU (SEQ ID NO: 1192) | | OMNI-171 |
| TRAC-S35 | GACCCUGCCGUGUACCAGC | GAGACTCT | OMNI-103, OMNI-120, |
| | UGA (SEQ ID NO: 1080) | | OMNI-125, OMNI-231, |
| | | | OMNI-247, OMNI-250, |
| | | | OMNI-281 |
| TRAC-S36 | UCAAAAUCGGUGAAUAGGC | CAGACTTG | OMNI-103, OMNI-120 |
| | AGA (SEQ ID NO: 1182) | | |
| TRAC-S40 | AUUCUGAUGUGUAUAUCAC | CAAAACTG | |
| | AGA (SEQ ID NO: 957) | | |
| TRAC-S41 | GCCUUCAACAACAGCAUUA | CAGAAGAC | OMNI-281 |
| | UUC (SEQ ID NO: 1929) | | |
| TRAC-S44 | GCCACUUUCAGGAGGAGGA | GGAACCCA | OMNI-131 |
| | UUC (SEQ ID NO: 1852) | | |
| TRAC-S56 | AUCCAGAACCCUGACCCUG | TGTACCAG | OMNI-131, OMNI-293, |
| | CCG (SEQ ID NO: 1924) | | OMNI-339 |
| TRAC-S58 | AGAACCCUGACCCUGCCGU | CCAGCTGA | OMNI-103 |
| | GUA (SEQ ID NO: 901) | | |
| TRAC-S59 | GUACCAGCUGAGAGACUCU | TCCAGTGA | OMNI-138 |
| | AAA (SEQ ID NO: 1138) | | |
| TRAC-S69 | UCCUGUGAUGUCAAGCUGG | AGAAAAGC | |
| | UCG (SEQ ID NO: 1422) | | |
| TRAC-S71 | GUCCCACAGAUAUCCAGAA | TGACCCTG | |
| | CCC (SEQ ID NO: 1930) | | |
| TRAC-S72 | UUCCAGAAGACACCUUCUU | CAGCCCAG | OMNI-269 |
| | CCC (SEQ ID NO: 1265) | | |
| TRAC-S74 | ACCUGUCAGUGAUUGGGUU | AATCCTCC | |
| | CCG (SEQ ID NO: 1767) | | |
| TRAC-S84 | UGGCCGGGUUUAAUCUGCU | GACGCTGC | OMNI-215 |
| | CAU (SEQ ID NO: 1899) | | |
| TRAC-S89 | CAUGAGGUCUAUGGACUUC | AGCAACAG | |
| | AAG (SEQ ID NO: 1001) | | |
| TRAC-S90 | UUCUGAUGUGUAUAUCACA | AAAACTGT | OMNI-103 |
| | GAC (SEQ ID NO: 1267) | | |
| TRAC-S91 | GCUGUGGCCUGGAGCAACA | CTGACTTT | OMNI-103 |
| | AAU (SEQ ID NO: 1120) | | |
| TRAC-S92 | UCCUCUUGUCCCACAGAUA | AGAACCCT | |
| | UCC (SEQ ID NO: 1931) | | |
| TRAC-S93 | AGCCGCAGCGUCAUGAGCA | TAAACCCG | |
| | GAU (SEQ ID NO: 1776) | | |
| TRAC-S95 | GAUAUCCAGAACCCUGACC | CCGTGTAC | OMNI-309, OMNI-322 |
| | CUG (SEQ ID NO: 1093) | | |
| TRAC-S96 | CUCAUGACGCUGCGGCUGU | CCAGCTGA | OMNI-127 |
| | GGU (SEQ ID NO: 1829) | | |
| TRAC-S101 | AACAGUGCUGUGGCCUGGA | ACAAATCT | OMNI-173 |
| | GCA (SEQ ID NO: 843) | | |
| TRAC-S106 | UGACAAGUCUGUCUGCCUA | ACCGATTT | |
| | UUC (SEQ ID NO: 1215) | | |
| TRAC-S115 | UGUGUAUAUCACAGACAAA | GTGCTAGA | |
| | ACU (SEQ ID NO: 1253) | | |
| TRAC-S124 | UCUCGACCAGCUUGACAUC | GGAACTTT | OMNI-103 |
| | ACA (SEQ ID NO: 1424) | | |
| TRAC-S128 | AGUCUCUCAGCUGGUACAC | AGGGTCAG | OMNI-218 |
| | GGC (SEQ ID NO: 930) | | |
| TRAC-S129 | GCAUGUGCAAACGCCUUCA | ACAGCATT | OMNI-127 |
| | ACA (SEQ ID NO: 1107) | | |
| TRAC-S135 | GUGUCACAAAGUAAGGAUU | ATGTGTAT | OMNI-308 |
| | CUG (SEQ ID NO: 1157) | | |
| TRAC-S142 | CGUCAUGAGCAGAUUAAAC | GCCACTTT | OMNI-103 |
| | CCG (SEQ ID NO: 1825) | | |
| TRAC-S144 | UUCUUCCCCAGCCCAGGUA | GCAGCTTT | OMNI-103 |
| | AGG (SEQ ID NO: 1932) | | |
| TRAC-S160 | CCCUGCCGUGUACCAGCUG | GACTCTAA | OMNI-266 |
| | AGA (SEQ ID NO: 1013) | | |
| TRAC-S168 | GAAGUCCAUAGACCUCAUG | AGCACAGT | OMNI-354 |
| | UCU (SEQ ID NO: 1070) | | |
| TRAC-S180 | CCUCCUCCUGAAAGUGGCC | TTTAATCT | |
| | GGG (SEQ ID NO: 1816) | | |
| TRAC-S184 | CUGGAUUUAGAGUCUCUCA | GGTACACG | |
| | GCU (SEQ ID NO: 1046) | | |

TABLE 10
Plasmid Constructs and Elements

| Plasmid | Elements | Coding ORF Sequence | Example | DNA Sequence |
|---|---|---|---|---|
| pET9a | Expressing OMNI | T7 promoter HA Tag- | pET9a-OMNI-103 | SEQ ID NO: 2083 |
| | polypeptide in the | Linker-OMNI ORF | | |
| | bacterial system | (Human optimized) - | | |
| | | SV40 NLS-8XHisTag - | | |
| | | T7 terminator | | |
| pbShuttle | Expressing OMNI | U6 promotor - | pShuttle Guide- | SEQ ID NO: 2084 |
| Guide T2 | sgRNA in the | T7promoter - T2 spacer | T2-OMNI-103 V2 | |
| | bacterial system | sgRNA scaffold - T7 | | |
| | | terminator | | |
| pbPOS T2 | Bacterial/TXTL | T2 protospacer - 8N | pbPOS T2 library | SEQ ID NO: 2085 |
| library | depletion assay | PAM library - | | |
| | | chloramphenicol | | |
| | | acetyltransferase | | |

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Anzalone et al. (2019) “Search-and-replace genomme editing without double-strand brakes or donor DNA”, Nature 576, 149-157.
4. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.
5. Behr (1994) Gene transfer with synthetic cationic amphiphiles: Prospects for gene therapy”, Bioconjuage Chem 5:382-89.
6. Blaese (1995) “Vectors in cancer therapy: how will they deliver”, Cancer Gene Ther. 2:291-97.
7. Blaese et al. (1995) “T lympocyte-directed gene therapy for ADA-SCID: initial trial results after 4 years”, Science 270(5235):475-80.
8. Buchschacher and Panganiban (1992) “Human immunodeficiency virus vectors for inducible expression of foreign genes”, J. Virol. 66:2731-39.
9. Burstein et al. (2017) “New CRISPR-Cas systems from uncultivated microbes”, Nature 542:237-41.
10. Chang and Wilson (1987) “Modification of DNA ends can decrease end-joining relative to homologous recombination in mammalian cells”, Proc. Natl. Acad. Sci. USA 84:4959-4963.
11. Chung et al. (2006) “Agrobacterium is not alone: gene transfer to plants by viruses and other bacteria”, Trends Plant Sci. 11(1):1-4.
12. Coelho et al. (2013) “Safety and efficacy of RNAi therapy for transthyretin amyloidosis” N. Engl. J. Med. 369, 819-829.
13. Collias and Beisel (2021) “CRISPR technologies and the search for the PAM-free nuclease”, Nature Communications 12, 555.
14. Crystal (1995) “Transfer of genes to humans: early lessons and obstacles to success”, Science 270(5235):404-10.
15. Dillon (1993) “Regulation gene expression in gene therapy” Trends in Biotechnology 11(5):167-173.
16. Dranoff et al. (1997) “A phase I study of vaccination with autologous, irradiated melanoma cells engineered to secrete human granulocyte macrophage colony stimulating factor”, Hum. Gene Ther. 8(1):111-23.
17. Dunbar et al. (1995) “Retrovirally marked CD34-enriched peripheral blood and bone marrow cells contribute to long-term engraftment after autologous transplantation”, Blood 85:3048-57.
18. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20.
19. Gao and Huang (1995) “Cationic liposome-mediated gene transfer” Gene Ther. 2(10):710-22.
20. Haddada et al. (1995) “Gene Therapy Using Adenovirus Vectors”, in: The Molecular Repertoire of Adenoviruses III: Biology and Pathogenesis, ed. Doerfler and Böhm, pp. 297-306.
21. Han et al. (1995) “Ligand-directed retro-viral targeting of human breast cancer cells”, Proc Natl Acad Sci U.S.A. 92(21): 9747-51.
22. Hu et al. (2018) “Evolved Cas9 variants with broad PAM compatibility and high DNA specificity”, Nature 556(7699):57-63.
23. Jinek et al. (2012) “A programmable dual-RNA-guided DNA endonuclease in adaptive bacterial immunity,” Science 337(6096): 816-21.
24. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
25. Judge et al. (2006) “Design of noninflammatory synthetic siRNA mediating potent gene silencing in vivo”, Mol Ther. 13(3):494-505.
26. Kohn et al. (1995) “Engraftment of gene-modified umbilical cord blood cells in neonates with adnosine deaminase deficiency”, Nature Medicine 1:1017-23.
27. Kremer and Perricaudet (1995) “Adenovirus and adeno-associated virus mediated gene transfer”, Br. Med. Bull. 51(1):31-44.
28. Macdiarmid et al. (2009) “Sequential treatment of drug-resistant tumors with targeted minicells containing siRNA or a cytotoxic drug”, Nat Biotehcnol. 27(7):643-51.
29. Malech et al. (1997) “Prolonged production of NADPH oxidase-corrected granulocyes after gene therapy of chronic granulomatous disease”, PNAS 94(22):12133-38.
30. Miller et al. (1991) “Construction and properties of retrovirus packaging cells based on gibbon ape leukemia virus”, J Virol. 65(5):2220-24.
31. Miller (1992) “Human gene therapy comes of age”, Nature 357:455-60.
32. Mitani and Caskey (1993) “Delivering therapeutic genes—matching approach and application”, Trends in Biotechnology 11(5): 162-66.
33. Nabel and Felgner (1993) “Direct gene transfer for immunotherapy and immunization”, Trends in Biotechnology 11(5):211-15.
34. Nishimasu et al. (2018) “Engineered CRISPR-Cas9 nuclease with expanded targeting space”, Science 361(6408): 1259-1262.
35. Remy et al. (1994) “Gene Transfer with a Series of Lipphilic DNA-Binding Molecules”, Bioconjugate Chem. 5(6):647-54.
36. Sentmanat et al. (2018) “A Survey of Validation Strategies for CRISPR-Cas9 Editing”, Scientific Reports 8:888, doi: 10.1038/s41598-018-19441-8.
37. Sommerfelt et al. (1990) “Localization of the receptor gene for type D simian retroviruses on human chromosome 19”, J. Virol. 64(12):6214-20.
38. Van Brunt (1988) “Molecular framing: transgenic animals as bioactors” Biotechnology 6:1149-54.
39. Vigne et al. (1995) “Third-generation adenovectors for gene therapy”, Restorative Neurology and Neuroscience 8(1,2):35-36.
40. Walton et al. (2020) “Unconstrained genome targeting with near-PAMless engineered CRISPR-Cas9 variants”, Science 368(6488):290-296.
41. Weissman and Kariko (2015) “mRNA:Fulfilling the promise of gene therapy”, Molecular Therapy (9):1416-7.
42. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
43. Yu et al. (1994) “Progress towards gene therapy for HIV infection”, Gene Ther. 1(1):13-26.
44. Zetsche et al. (2015) “Cpfl is a single RNA-guided endonuclease of a class 2 CRIPSR-Cas system” Cell 163(3):759-71.
45. Zuris et al. (2015) “Cationic lipid-mediated delivery of proteins enables efficient protein based genome editing in vitro and in vivo” Nat Biotechnol. 33(1):73-80.

1. A method for inactivating alleles of the T Cell Receptor Alpha Constant (TRAC) gene in a cell, the method comprising
introducing to the cell a composition comprising:
at least one CRISPR nuclease, or a sequence encoding a CRISPR nuclease; and
an RNA molecule comprising a guide sequence portion,
wherein a complex of the CRISPR nuclease and the RNA molecule affects a double strand break in alleles of the TRAC gene,
wherein the guide sequence portion of the RNA molecule comprises 17-50 contiguous nucleotides containing nucleotides in the sequence set forth in any one of SEQ ID NOs: 1-1932.

2. The method of claim 1, wherein the composition is introduced to a cell in a subject or to a cell in culture.

3. The method of any one of claims 1-2, wherein the cell is a lymphocyte, a T cell, a T regulatory cell, a stem cell, or a fibroblast, blood cell, hepatocyte, keratinocyte, or any other cell type capable of being reprogrammed to an induced pluripotent stem cell (iPSC).

4. The method of any one of claims 1-2, wherein the cell is a hematopoietic stem cell (HSC), induced pluripotent stem cell (iPS cell), iPSc-derived cell, natural killer cell (NK), iPS-derived NK cell (iNK), T cell, innate-like T cell (iT), natural killer T cell (NKT), γ δ T cell, iPSc-derived T cell, invariant NKT cells (iNKT), iPSc-derived NKT, monocyte, or macrophage.

5. The method of any one of claims 1-4, wherein the CRISPR nuclease and the RNA molecule are introduced to the cell at substantially the same time or at different times.

6. The method of any one of claims 1-5, wherein alleles of the TRAC gene in the cell are subjected to an insertion or deletion mutation.

7. The method of claim 6, wherein the insertion or deletion mutation creates an early stop codon.

8. The method of any one of claims 1-7, wherein the inactivating results in a truncated protein encoded by the mutated allele.

9. The method of any one of claims 1-8, wherein the composition introduced to the cell further comprises a second RNA molecule comprising a guide sequence portion, wherein the guide sequence portion of the RNA molecule comprises 17-50 contiguous nucleotides containing nucleotides in the sequence set forth in any one of SEQ ID NOS: 1-1932.

10. A method for inactivating alleles of the T Cell Receptor Alpha Constant (TRAC) gene in a cell, the method comprising
introducing to the cell a composition comprising:
at least one CRISPR nuclease, or a sequence encoding a CRISPR nuclease; and
an RNA molecule comprising a guide sequence portion,
wherein a complex of the CRISPR nuclease and the RNA molecule affects a double strand break in alleles of the TRAC gene,
wherein the guide sequence portion of the RNA molecule comprises 17-50 contiguous nucleotides containing nucleotides in the sequence set forth in any one of SEQ ID NOs: 1-1932 modified to contain 1, 2, 3, 4, or 5 nucleotide mismatches relative to a fully-complementary target sequence of the guide sequence portion.

11. The method of claim 10, wherein the guide sequence portion of the RNA molecule comprises 1, 2, 3, 4, or 5 nucleotide mismatches relative to a fully-complementary target sequence of the guide sequence portion.

12. The method of any one of claim 10 or 11, wherein the guide sequence portion provides higher targeting specificity to the complex of the CRISPR nuclease and the RNA molecule relative to a guide sequence portion that has higher complementarity to an allele of the TRAC gene.

13. The method of any one of claims 10-12, wherein the composition introduced to the cell further comprises a second RNA molecule comprising a guide sequence portion, wherein the guide sequence portion of the RNA molecule comprises 17-50 contiguous nucleotides containing nucleotides in the sequence set forth in any one of SEQ ID NOs: 1-1932, or any one of SEQ ID NOs: 1-1932 modified to contain 1, 2, 3, 4, or 5 nucleotide mismatches relative to a fully-complementary target sequence of the guide sequence portion.

14. A composition comprising an RNA molecule which comprises 17-50 contiguous nucleotides containing nucleotides in the sequence set forth in any one of SEQ ID NOs: 1-1932.

15. The composition of claim 14, wherein the RNA molecule comprises 17-50 contiguous nucleotides containing nucleotides in the sequence set forth in any one of SEQ ID NOs: 459, 482, 499, 608, 696, 719, 724, 1590, 1596, 1620, 1671, 1673, 1690 and 1723.

16. The composition of claim 14, wherein the RNA molecule comprises 17-50 contiguous nucleotides containing nucleotides in the sequence set forth in any one of SEQ ID NOs: 1-1932 other than SEQ ID NOs: 459, 482, 499, 608, 696, 719, 724, 1590, 1596, 1620, 1671, 1673, 1690 and 1723.

17. The composition of any one of claims 13-15, further comprising a second RNA molecule which comprises 17-50 contiguous nucleotides containing nucleotides in the sequence set forth in any one of SEQ ID NOs: 1-1932, or any one of SEQ ID NOs: 1-1932 modified to contain 1, 2, 3, 4, or 5 nucleotide mismatches relative to a fully-complementary target sequence of the guide sequence portion.

18. The composition of claim 17, wherein the second RNA molecule which comprises 17-50 contiguous nucleotides containing nucleotides in the sequence set forth in any one of SEQ ID NOs: 459, 482, 499, 608, 696, 719, 724, 1590, 1596, 1620, 1671, 1673, 1690 and 1723.

19. The composition of claim 17, wherein the second RNA molecule which comprises 17-50 contiguous nucleotides containing nucleotides in the sequence set forth in any one of SEQ ID NOs: 1-1932 is other than SEQ ID NOs: 459, 482, 499, 608, 696, 719, 724, 1590, 1596, 1620, 1671, 1673, 1690 and 1723.

20. A composition comprising an RNA molecule which comprises 17-50 contiguous nucleotides containing nucleotides in the sequence set forth in any one of SEQ ID NOS: 1-1932, or any one of SEQ ID NOs: 1-1932 modified to contain 1, 2, 3, 4, or 5 nucleotide mismatches relative to a fully-complementary target sequence of the guide sequence portion.

21. The composition of claim 20, further comprising a second RNA molecule which comprises 17-50 contiguous nucleotides containing nucleotides in the sequence set forth in any one of SEQ ID NOs: 1-1932, or any one of SEQ ID NOs: 1-1932 modified to contain 1, 2, 3, 4, or 5 nucleotide mismatches relative to a fully-complementary target sequence of the guide sequence portion.

22. The composition of any one of claims 14-21, further comprising a CRISPR nuclease.

23. The composition of any one of claims 14-22, further comprising a tracrRNA molecule.

24. A cell modified by the method of any one of claims 1-13 or modified using the composition of any one of claims 14-23.

25. The modified cell of claim 24, wherein the cell is any one of a wherein the cell is a lymphocyte, a T cell, a T regulatory cell, a stem cell, or a fibroblast, blood cell, hepatocyte, keratinocyte, or any other cell type capable of being reprogrammed to an induced pluripotent stem cell (iPSC).

26. The modified cell of claim 24, wherein the cell is a hematopoietic stem cell (HSC), induced pluripotent stem cell (iPS cell), iPSc-derived cell, natural killer cell (NK), iPS-derived NK cell (iNK), T cell, innate-like T cell (iT), natural killer T cell (NKT), γ δ T cell, iPSc-derived T cell, invariant NKT cell (iNKT), iPSc-derived NKT, monocyte, or macrophage.

27. The modified cell of claim 24, wherein the cell is a stem cell or any cell type capable of being reprogrammed to an induced pluripotent stem cell (iPSC).

28. The modified cell of claim 27, wherein the stem cell is differentiated after it is modified.

29. The modified cell of claim 28, wherein the stem cell is differentiated into any one of a lymphocyte, a T cell, a T regulatory cell, a B cell, a natural killer (NK) cell, innate-like T cell (iT), natural killer T cells (NKT), γ δ T cell, invariant NKT cells (iNKT), monocyte, or macrophage.

30. A medicament comprising the composition of any one of claims 14-23 for use in inactivating a TRAC allele in a cell, wherein the medicament is administered by delivering to the cell the composition of any one of claims 14-23.

31. Use of the composition of any one of claims 14-23 or the modified cell of any one of claims 24-29 for treating, ameliorating, or preventing graft-versus-host-disease (GVHD), comprising delivering the composition of any one of claims 14-23 or the modified cell of any one of claims 24-29 to a subject experiencing or at risk of experiencing graft-versus-host-disease (GVHD).

32. A medicament comprising the composition of any one of claims 14-23 or the modified cell of any one of claims 24-29 for use in treating, ameliorating, or preventing graft-versus-host-disease (GVHD), wherein the medicament is administered by delivering the composition of any one of claims 14-23 or the modified cell of any one of claims 24-29 to a subject experiencing or at risk of experiencing graft-versus-host-disease (GVHD).

33. A kit for inactivating a TRAC allele in a cell, comprising the composition of any one of claims 14-23 and instructions for delivering the composition to the cell.

34. The kit of claim 33, wherein the composition is delivered to the cell ex vivo.

35. A kit for treating or preventing graft-versus-host-disease (GVHD) in a subject, comprising the composition of any one of claims 14-23 or the modified cell of any one of claims 24-29 and instructions for delivering the composition to a subject experiencing or at risk of experiencing graft-versus-host-disease (GVHD).
